(19) United States
(12) Patent Application Publication
(10) Pub. No.: US 2004/0093654 A1
(43) Pub. Date: May 20, 2004

(54) Eye shield/shade apparatus for attachment to caps and eyeglasses

(76) Inventors: Terrence M. Hanrahan, Mission Viejo, CA (US); Willard J. Hall, La Puente, CA (US); Bill R. Hall, Brea, CA (US); Stephen L. Burd, Anaheim, CA (US)

Correspondence Address: SNELL & WILMER, ONE ARIZONA CENTER, 400 EAST VAN BUREN, PHOENIX, AZ, 850040001

(21) Appl. No.: 10/295,508

(22) Filed: Nov. 15, 2002

(51) Int. Cl. A61F 9/00

(52) U.S. Cl. 2/12

(57) ABSTRACT

An eyeshield/eyeshade attachment apparatus for attachment to the bill of a cap or visor that is capable of axial parallel positioning with respect to the bill of the cap or visor is presented. The apparatus includes a clip member, a support member attached to the clip member, a shield attached to the support member, and means for engaging the clip member with the support member such that the shield can rotate up to 180 degrees in relation to the clip member.

### FIELD OF INVENTION

The present invention generally relates to an eye shield/shade apparatus for attachment to a baseball style cap or hat and all types of eyeglasses, and more particularly, to a sun shield apparatus for attachment to a baseball style cap or hat and all types of eyeglasses which includes at least one clip member having a hollow member attached to an outer surface of the clip member, a shield, and a bar member attached to the shield where a portion of the bar member is contained within the hollow member of the clip member such that the shade member can rotate up to 180 degrees in relation to the clip member.

### BACKGROUND OF THE INVENTION

A variety of eye shields and eyeshades designed for attachment to the brim or bill of a baseball style cap, as well as for attachment to eyeglasses, are known in the art. However, most existing eye shield and eyeshade attachments are designed to move from an inoperable position to an operable position which spans an angle of up to ninety degrees from the inoperable position thereby providing a shade or shield that is axially perpendicular with respect to the brim or bill of a hat or the frame of a pair of eyeglasses.

For example, U.S. Pat. No. 5,687,420 issued to Chong discloses a device for affixing a sunshade to a cap which includes a pair of sunshades fixed to an upper rib having exposed left and right side portions, and left and right side anchors affixed to the brim of the cap having clip elements that clip onto exposed portions of the upper rib of the sunshades to hold the sunshades in place. The upper rib and clip elements include cam members and cam surfaces, respectively, for maintaining the sunshade in an operative position or storage position as shown in FIG. 3 of the patent.

U.S. Pat. No. 5,689,827, issued to Ryder, further illustrates an eyeshield attached to the underside of a cap visor. The eyeshield is pivotally coupled to the connector to enable its movement from an inoperative position parallel to the visor to an operative position perpendicular to the visor and extending downward toward the ground from underneath the visor. In addition, U.S. Pat. No. 5,615,413, issued to Bower, describes an eyeshield for removable attachment to a visor or cap bill which uses clips for attachment to the visor or cap. The eyeshield attachment includes anchors that can be removably attached to the clips by hinge pins that protrude downward through the clips. The anchors are attached to the eyeshield which is flipped down for operative use and flipped upward against the underside of the visor or bill when not in use. Like the previously described patent references, this reference discloses an eyeshield or eyeshade that is capable of axially perpendicular movement with respect to a visor or bill of a cap.

Prior art eyeshield and eyeshade attachments are typically stored underneath a visor or bill of a cap when not being used in an operable position. This creates a visible obstruction in a user's direct line of sight when moving the eyeshield or eyeshade in and out of the operable position that is close to the user's eyes. Moreover, storing the eyeshield or eyeshade so close to the user's eyes may create a safety hazard in the event that the attachment piece for the eyeshield or eyeshade becomes loose and disconnects, or in the event that the eyeshield or eyeshade is struck by another object and dislodged or shaken loose.

Further, prior art eyeshield and eyeshade attachments are not configured to allow for axially parallel positioning with respect to the bill of a cap or visor to enable a lateral extension of the bill. Laterally extending the bill of a cap or visor creates additional shade for the user by widening the bill of the cap or visor.

Accordingly, there is a need for an eyeshield or eyeshade for attachment to a cap or visor that is capable of being stored in a nonobtrusive location when it is not being used, and in a position that is directed away from a user's eyes. There is also a need for an eyeshield or eyeshade that is capable of being axially positioned parallel to the bill of a cap or visor and that is simple and cost effective to produce.

### SUMMARY OF THE INVENTION

The present invention is directed to an eye shield/shade attachment that can be removably attached to the bill of a baseball style cap or visor which is capable of axially positioning the eye shield/shade parallel to the bill, and capable of storing the eye shield/shade in a nonobtrusive position while attached to the cap or visor when the eye shield/shade is not in use. The eye shield/shade attachment includes a shade member, a support member connected to the shade member, and at least one clip member attached to the support member so that the shade member can rotate up to 180 degrees in relation to the clip member.

In another aspect of the invention, the clip member includes a hollow member attached to an outer surface of the clip member so that a portion of the support member is contained within the hollow member. The hollow member may be generally cylindrical in shape with an interior that includes multiple planar sides. The part of the support member contained within the hollow member is a generally cylindrical shaped end having a notch which traverses the diameter of the cylindrical shaped end and a pair of barbs with each barb being located on an opposite side of the notch. Each barb comprises a planar side for engaging a planar side in the interior of the hollow member. The cylindrical shaped end of the support member then rotates within the hollow member with the interior planar sides when the shade member is moved from an inoperable position to an operable position. Engagement of the barbs of end of the support member with pairs of oppositely disposed planar sides contained within the interior of the hollow member can provide a means for creating lock steps for maintaining the shade member at various positions with respect to the clip member.

In another aspect of the invention, the clip member may include at least one ridge on its interior surface to ensure a secure fit with the bill of a cap or visor, or the frame of a pair of eyeglasses. Further, the clip member may contain a plurality of oppositely disposed ridge members on its interior surface.

In another aspect of the invention, the shield member may include at least one contoured end to accommodate the curve of the bill of a cap or visor, or an arm of a pair of eyeglass frames.

### BRIEF DESCRIPTION OF THE DRAWINGS

Additional aspects of the present invention should become evident upon reviewing the non-limiting embodiments described in the specification taken in conjunction with the accompanying figures, wherein like numerals designate like elements, and:

FIG. 1 is a perspective view of the eyeshield attachment of the present invention with the shield shown in a stored, nonoperative position;

FIG. 2 is a perspective view of the eyeshield attachment of the present invention with the shield shown in an operative position;

FIG. 3 is a magnified cross sectional view of the hollow member that is attached to the clip member (not shown);

FIG. 4 is a magnified side elevational view of an end of the support member having a slot or notch contained therein;

FIG. 5 is a side elevational view of the eyeshield attachment of the present invention shown with the shield in a stored, non-operative position;

FIG. 6 is a side elevational view of the eyeshield attachment of the present invention shown with the shield positioned at approximately a 45 degree angle with respect to the clip member;

FIG. 7 is a side elevational view of the eyeshield attachment of the present invention shown with the shield in an operative position at approximately a 90 degree angle with respect to the clip member;

FIG. 8 is a side elevational view of the eyeshield attachment of the present invention shown with the shield positioned at approximately a 180 degree angle with respect to the clip member; and

FIG. 9 is a perspective view of the eyeshield attachment of the present invention shown attached to the bill of a baseball cap with the shield in an operative position at approximately a 90 degree angle with respect to the clip member.

### DETAILED DESCRIPTION

The following descriptions are of exemplary embodiments of the invention only, and are not intended to limit the scope, applicability or configuration of the invention in any way. Rather, the following description is intended to provide a convenient illustration for implementing various embodiments of the invention. As will become apparent, various changes may be made in the function and arrangement of the elements described herein without departing from the spirit and scope of the invention. For example, though not specifically described, many shapes and orientations of the shield and/or support member, as well as the positioning of the support member in relation to the shield, should be understood to fall within the scope of the present invention.

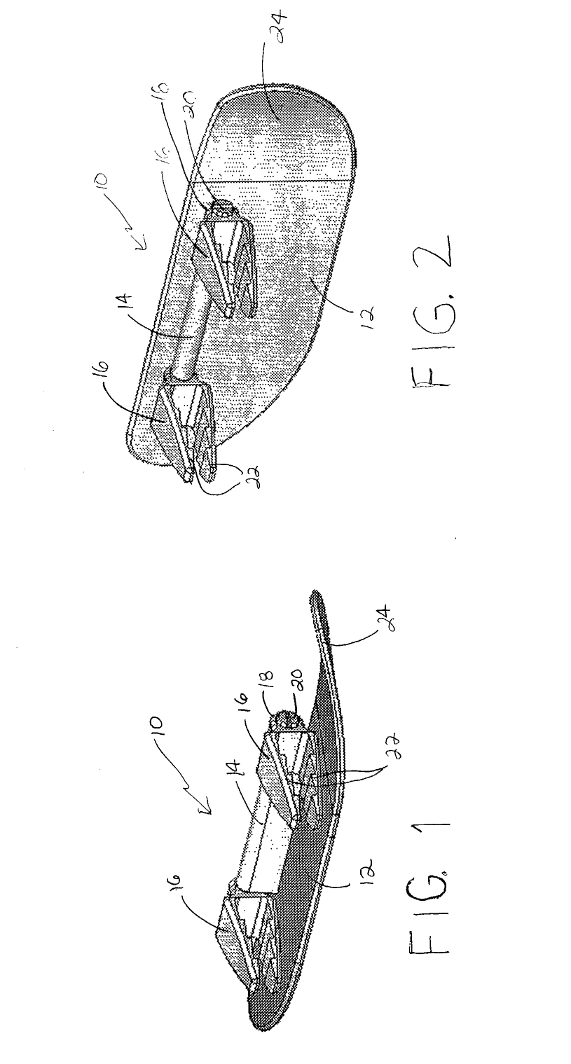

As shown in FIG. 1, the present invention is generally directed to an eyeshield or eyeshade attachment 10 which includes a shade or shield member 12, a support member 14 attached to shield 12, and at least one clip member 16 attached to shield 12. Clip member 16 comprises at least one hollow member 18 for engaging support member 14. At least one end 20 of support member 14 is contained within hollow member 18 so that end 20 of support member 14 rotates within hollow member 18 when shield 12 is moved from an operable position to an inoperable position, and vice versa.

Clip member 16 may further comprise at least one ridge 22, as shown in FIGS. 1 and 2, for assisting in securing eyeshield attachment 10 to the bill of a cap or visor, or even to the frame of a pair of eyeglasses. More preferably, clip member 16 may comprise pairs of oppositely disposed ridges 22 as shown in FIGS. 1 and 2 to further ensure the secure attachment of eyeshield attachment 10.

In use, clip members 16 shown in FIGS. 1 and 2 are attached to the bill of a cap or visor (not shown). FIG. 1 illustrates shield 12 of eyeshield attachment 10 in an inoperative or stored position. In this position, shield 12 is rotated until it is in a position adjacent to the underside of the bill of a cap or visor. In contrast, FIG. 2 illustrates shield 12 of eyeshield attachment 10 in an operable position. In this position, shield 12 is rotated until it is in a position that is perpendicular to the bill of a cap or visor, thereby shielding a user's eyes with shield 12.

Shield 12 is generally comprised of a plastic, polymer, cardboard, fiberboard, fabric, nylon, or any other material that is durable and stiff enough to hold its shape. Further, shield 12 may comprise a variety of shapes and configurations. For example, the embodiment of shield 12 shown in FIGS. 1 and 2 comprises a somewhat oblong shape, but further comprises a bent edge 24 which is designed to accommodate the curve in the bill of a cap or visor when eyeshield attachment 10 is attached to the bill of a cap or visor.

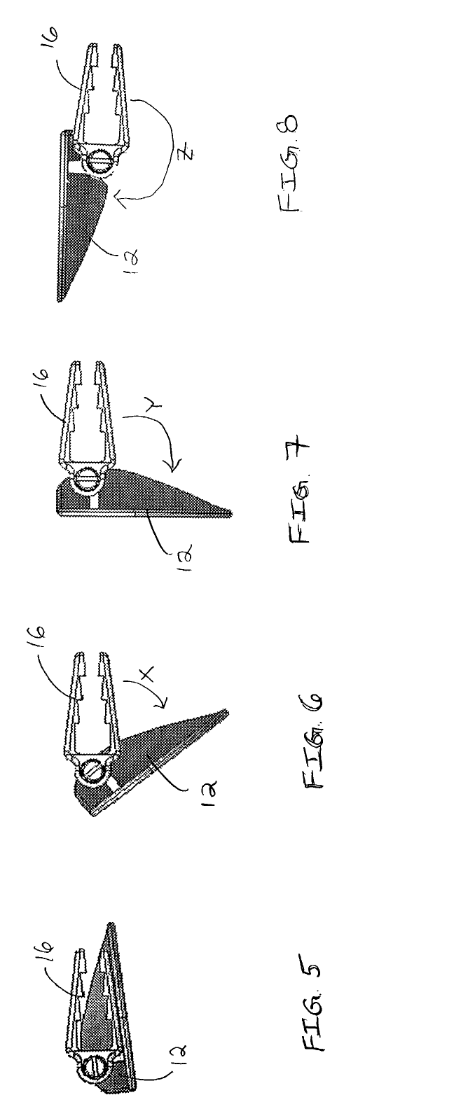

Support member 14 and clip member 16, which includes hollow member 18, are generally comprised of the same material as shield 12 described above, but is preferably comprised of a plastic or polymer. Like shield 12, the shape and configuration of both support member 14 and clip member 16 may vary considerably but must allow for at least a 180 degree rotation of shield 12 in relation to clip member 16. This allows for shield 12 to have multiple operable and inoperable positions which are later described with reference to FIGS. 5-8.

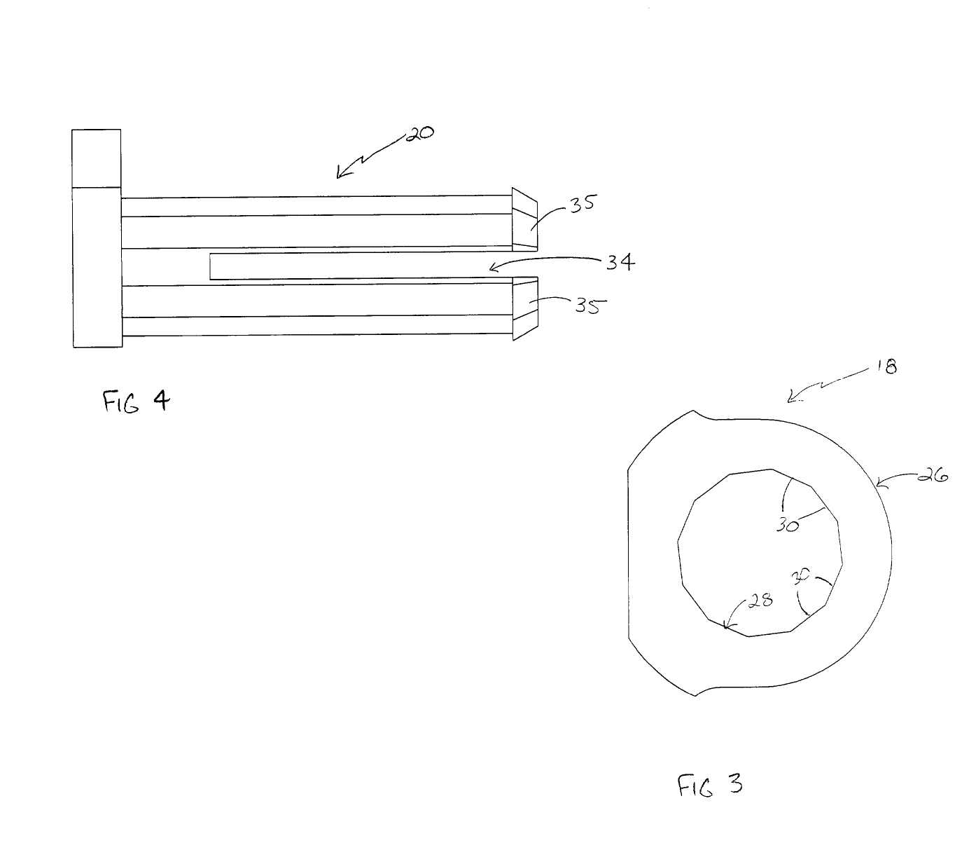

Turning now to FIG. 3, a magnified cross-sectional view of hollow member 18 of clip member 16 is shown. Hollow member 18 has an exterior surface 26 and an interior surface 28. In this exemplary embodiment, exterior surface 26 is generally cylindrical in shape and interior surface 28 comprises a plurality of planar sides 30 which extend around the entire interior surface 28 of hollow member 18.

A magnified side elevational view of an end 20 of support member 14 is illustrated in FIG. 4. In this exemplary embodiment, end 20 comprises a generally cylindrical shape having a slot 34 cut into end 20 which traverses the diameter of end 20 of support member 14. A pair of barbs 35, each having a planar side, are positioned on opposite sides of slot 34. As shown in eyeshield attachment 10 depicted in FIGS. 1-2, end 20 of support member 14 is contained in hollow member 18 of clip member 16. In order to move shield 12, which is attached to support member 14, a user can either apply pressure to shield 12 to push it outward and upward, or apply pressure to shield 12 to push it in a downward direction. When shield 12 is pushed in either a clockwise or counterclockwise direction, end 20 of support member 14 rotates within the interior of hollow member 18 of clip member 16. Planar sides 30 on interior surface 28 of hollow member 18 aid in forming small lock steps while moving the position of shield 12 by aligning with the planar edges of barbs 35 positioned on opposite sides of slot 34 in end 20.

FIGS. 5-8 illustrate side elevational views of the eyeshield attachment of the present invention shown in various operative and inoperative positions. FIG. 5 is a side elevational view of eyeshield apparatus 10 shown with shield 12 in a stored, inoperative position. Clip members 16 would be secured to the bill of a cap or visor (not shown) and shield 12 would lie adjacent to the underside of the bill of the cap or visor. When shield 12 is moved out of this stored position, shield 12 is pushed slightly downward at first as shown in FIG. 6 by arrow X. FIG. 6 is a side elevational view of eyeshield attachment 10 of the present invention shown with shield 12 positioned at approximately a 45 degree angle with respect to clip member 16. Depending upon a user's needs, eyeshield attachment 10 may be attached to the bill of a user's cap and worn in this position particularly if shield 12 is being used to block the sun when the sun is at a specific point in the sky where such a position is the most successful at blocking sunlight.

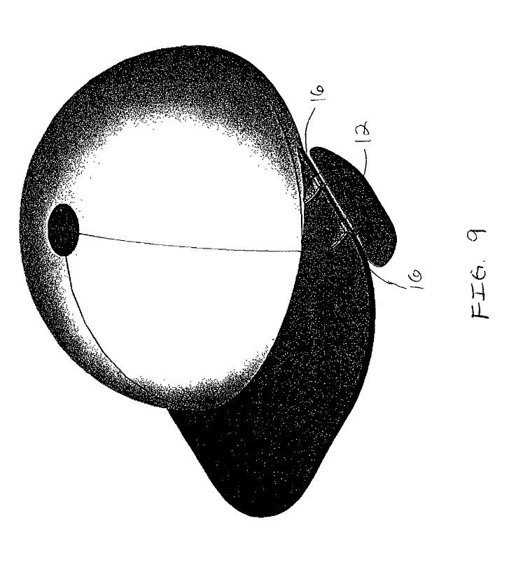

Shield 12 can then be pushed outward as shown by arrow Y to arrive at a position shown in FIG. 7. FIG. 7 is a side elevational view of eyeshield attachment 10 shown with shield 12 in an operative position at approximately a 90 degree angle with respect to clip member 16. If shield 12 is then pushed upward as shown by arrow Z, shield 12 arrives at a position that is parallel with clip member 16. FIG. 8 is a side elevational view of eyeshield attachment 10 with shield 12 shown positioned at approximately a 180 degree angle with respect to clip member 16. This position of shield 12 in effect widens the bill of a cap or visor depending upon the placement of eyeshield attachment, 10 on the bill of the cap or visor.

FIG. 9 shows the eyeshield attachment 10 of the present invention secured to the bill of a baseball cap in an operative position. FIG. 9 is a perspective view of the eyeshield attachment of the present invention shown attached to the bill of a baseball cap such that shield 12 is in an operative position at approximately a 90degree angle with respect to clip member 16.

The ability for shield 12 to rotate up to 180 degrees with respect to clip member 16 is an improvement over the prior art in that it provides a shield attachment with a wider range of operative positions thereby increasing the effectiveness of the shield attachment.

Thus, it should be apparent that many alterations of the eyeshield attachment apparatus described herein may be made. For example, as mentioned above, the shape and configuration of shield 12 may vary. Further, clip member 16 and support member 14 may vary in configuration and shape as long as their engagement allows for shield 12 to rotate up to 180 degrees in relation to clip member 16.

Lastly, various aspects of the invention have been described in illustrative embodiments. Of course, many combinations and modifications of the above-described structures, arrangements, proportions, elements, materials and components, used in the practice of the invention, in addition to those not specifically described, may be varied and particularly adapted to specific environments and operating requirements without departing from those principles.

1. An eyeshade for attachment to a cap or a pair of eyeglasses comprising:
a shade member;
a support member connected to said shade member; and
at least one clip member connected to said support member such that said clip member is capable of rotating up to 180 degrees in relation to said shade member.

2. The eyeshade of claim 1 wherein said clip member comprises a hollow member attached to an outer surface of said clip member such that at least a portion of said support member is contained within said hollow member.

3. The eyeshade of claim 2 wherein said hollow member is generally cylindrical in shape with an interior surface comprising a plurality of planar sides.

4. The eyeshade of claim 3 wherein the portion of said support member contained within said hollow member is an end of said support member comprising a generally cylindrical shape, a notch which traverses a diameter of the end of said support member, and a pair of barbs positioned on opposite sides of said notch.

5. The eyeshade of claim 1 wherein said clip member comprises at least one ridge located on an interior surface of said clip member.

6. The eyeshade of claim 5 wherein said clip member comprises a plurality of oppositely disposed ridges located on an interior surface of said clip member.

7. A eyeshield attachment apparatus comprising:
a shield;
at least one clip member having a hollow member attached to an outer surface of the clip member; and
a bar member attached to the shield wherein at least a portion of the bar member is contained within the hollow member of the clip member such that said clip member can rotate about said bar member up to 180 degrees in relation to said shield.

8. The eyeshield attachment apparatus of claim 7 wherein an interior surface of said hollow member comprises a plurality of planar sides.

9. The eyeshield attachment apparatus of claim 7 wherein the portion of said bar member contained within the hollow member comprises a slot traversing the diameter of the bar member and a pair of barbs positioned on opposite sides of said slot.

10. The eyeshield attachment apparatus of claim 7 wherein the clip member further comprises at least one ridge located on an interior surface of said clip member.

11. The eyeshield attachment apparatus of claim 10 wherein said clip member comprises a plurality of oppositely disposed ridges located on an interior surface of said clip member.

12. The eyeshield attachment apparatus of claim 7 having two clip members wherein said bar member comprises first and second ends and wherein each of said ends is contained within the hollow member of one of said two clip members.

13. The eyeshield attachment apparatus of claim 12 wherein a middle portion of said bar member is attached to the shield.

14. The eyeshield attachment apparatus of claim 12 wherein an interior surface of said hollow members comprises a plurality of planar sides.

15. The eyeshield attachment apparatus of claim 14 wherein each end of said bar member contained within the hollow members of the two clip members comprises a slot traversing the diameter of the bar member and a pair of barbs located on opposite sides of the slot

16. The eyeshield attachment apparatus of claim 7 wherein said shield comprises at least one contoured end to accommodate the curve of a bill of a cap.

17. A method for improving shade and/or protection of a user's eye when wearing a cap or visor having a bill comprising the steps of:
providing an eyeshield attachment having a clip member and a shade member attached to the clip member where the clip member is capable of rotating up to 180 degrees with respect to the shade member;
clipping the clip member to the bill of the cap or visor; and
adjusting the position of the shade member in relation to the clip member to
provide additional shade and/or protection to the user's eye.

18. The method of claim 17 further comprising the step of storing the shade member when not in use by adjusting the position of the shade member so that it is in a position that is either approximately 0 degrees or 180 degrees in relation to the clip member.

19. A method for making an eyeshield/eyeshade for attachment to a bill of a cap or visor comprising the steps of:
providing a shade member;
connecting a support member to the shade member; and
connecting at least one clip member to the support member such that said clip member is capable of rotating up to 180 degrees in relation to said shade member.

20. The method of claim 19 wherein said method of connecting said at least one clip member to the support member comprises the step of inserting a portion of said support member in a hollow portion of said clip member.
